(19) United States
(12) Patent Application Publication
(10) Pub. No.: US 2011/0015565 A1
(43) Pub. Date: Jan. 20, 2011

(54) GAS DISPENSER WITH THERAPEUTIC AGENT

(76) Inventor: Francis X. Hursey, West Hartford, CT (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/503,481

(22) Filed: Jul. 15, 2009

(51) Int. Cl. A61M 37/00

(52) U.S. Cl. 604/24

(57) ABSTRACT

Various embodiments described herein promote accelerated healing of a wound. For example, in some embodiments, a gas emitter, such as an oxygen emitter, and a therapeutic agent, such as a zeolite, are positioned proximal to a wound. In some embodiments, the therapeutic agent is dispersed or contained in a substrate or pouch removably attached to the gas emitter. The therapeutic agent can be a topical agent configured to make contact with the wound during therapy.

### BACKGROUND OF THE INVENTION

1. Field of the Invention

This disclosure relates generally to wound therapy and, more particularly, to devices and methods for wound treatment using gas-emitting therapy and one or more therapeutic agents.

2. Description of the Related Art

In some circumstances, a patient's body is unable to heal a wound on its own. Many factors can cause a particular wound to become a hard-to-heal wound, such as the size and severity of the wound, the patient's age, illness, the location of the wound, the nutritional intake of the patient, etc. One situation in which hard-to-heal wounds can appear is when a patient is in a laying position for an extended period of time. If not moved frequently enough, the patient can develop pressure ulcers (e.g., bedsores). Many bedsores can develop into hard-to-heal wounds. In some circumstances, excessive or stubborn bleeding can prevent a wound from healing. Thus, in some circumstances, stopping bleeding from a wound is a priority not only to prevent blood loss but also to enable the healing process to proceed unimpeded.

Even in wounds that will eventually heal without medical attention, it can be desirable to accelerate the healing process. The longer a wound takes to heal, the greater the risk of infection. It is also often desirable to improve the wound healing process by improving the quality of the new tissue and reducing the amount of scaring that remains after a wound heals.

### SUMMARY OF THE INVENTION

Embodiments described herein have several features, no single one of which is solely responsible for their desirable attributes. Various embodiments described herein promote accelerated healing of a wound. For example, in some embodiments, a gas emitter, such as an oxygen emitter, and a therapeutic agent, such as a zeolite, are positioned proximal to a wound. In some embodiments, the therapeutic agent is dispersed or contained in a substrate or pouch removably attached to the gas emitter. The therapeutic agent can be a topical agent configured to make contact with the wound during therapy.

Some embodiments disclosed herein include a device for treating a wound. In some embodiments the device includes a gas permeable pad configured to be placed in proximity to a wound, and the gas permeable pad may include a therapeutic agent. The device can also include a gas dispenser disposed adjacent to the gas permeable pad, and the gas dispenser can be configured to direct gas through the gas permeable pad.

In some embodiments, the gas dispenser can be configured to direct a therapeutic concentration of oxygen through the gas permeable pad. The gas permeable pad can include a material or portion that is permeable to the gas. The gas permeable pad can include a plurality of holes extending therethrough to allow gas to pass through the gas permeable pad.

In some embodiments, the therapeutic agent can be a molecular sieve material. The therapeutic agent can include zeolite particles. For example, the zeolite particles can be granular zeolite particles, and in some embodiments can have an average size greater than or equal to about 0.4 mm and/or less than or equal to about 2.4 mm. The zeolite particles can have an average moisture content greater than or equal to about 5% and/or less than or equal to about 15% by weight. The therapeutic agent can be applied to a surface of the gas permeable pad configured to face the wound.

In some embodiments, the gas dispenser can include an inlet configured to connect to an external gas supply. The gas dispenser can include a plurality of outlets and one or more distribution structures contained within the gas dispenser, and the distribution structures can be configured to affect the distribution of the gas through the outlets.

The gas dispenser may also include an inlet for taking in atmospheric air and an oxygen generator contained within the gas dispenser. The oxygen generator can be configured to extract the oxygen from the atmospheric air and direct the oxygen toward the gas permeable pad.

In some embodiments, the gas permeable pad can be removably attachable to the gas dispenser. The gas permeable pad can be secured to the gas dispenser. In some embodiments, one or more portions of the device can be a single-use disposable unit.

The gas dispenser may include a flexible material and one or more support structures disposed inside the gas dispenser. The support structures can be configured to prevent the gas dispenser from collapsing when pressure is applied to the gas dispenser.

Some examples of methods of treating a wound on a patient are disclosed. Some methods can include applying a gas permeable pad to the wound. The gas permeable pad can include a therapeutic agent, and the therapeutic agent can be configured to be placed in direct contact with the wound. The method can also include dispersing gas through the gas permeable pad and onto the wound.

In some embodiments, the gas can include a therapeutic concentration of oxygen, the wound can be a non-bleeding wound, and/or the therapeutic agent can include zeolite particles. In some embodiments, applying a gas permeable pad to the wound includes placing the gas permeable pad directly under a portion of the patient such that the weight of the patient presses the therapeutic agent into contact with the wound.

### BRIEF DESCRIPTION OF THE DRAWINGS

The following drawings and the associated descriptions are provided to illustrate embodiments of the present disclosure and do not limit the scope of the claims.

FIG. 1 schematically shows an example of a wound treatment system having a gas dispenser and a pad incorporating a therapeutic agent.

FIG. 2 is a cross-sectional view of the gas dispenser and pad of FIG. 1.

FIG. 3 is a cross-sectional view of an embodiment of a wound treatment device.

FIG. 4 is a cross-sectional view of an embodiment of a wound treatment device having an oxygen generator.

FIG. 5 schematically shows features of an example of a gas generator.

FIG. 6 is a flowchart showing an exemplary embodiment of a method for treating a wound.

### DETAILED DESCRIPTION OF THE PREFERRED EMBODIMENT

Embodiments of the inventions will now be described with reference to the accompanying figures. Although certain preferred embodiments and examples are disclosed herein, inventive subject matter extends beyond the specifically disclosed embodiments to other alternative embodiments and/or uses of the inventions, and to modifications and equivalents thereof. Thus, the scope of the inventions herein disclosed is not limited by any of the particular embodiments described below. For example, in any method or process disclosed herein, the acts or operations of the method or process may be performed in any suitable sequence and are not necessarily limited to any particular disclosed sequence.

Certain aspects and advantages of the disclosed embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

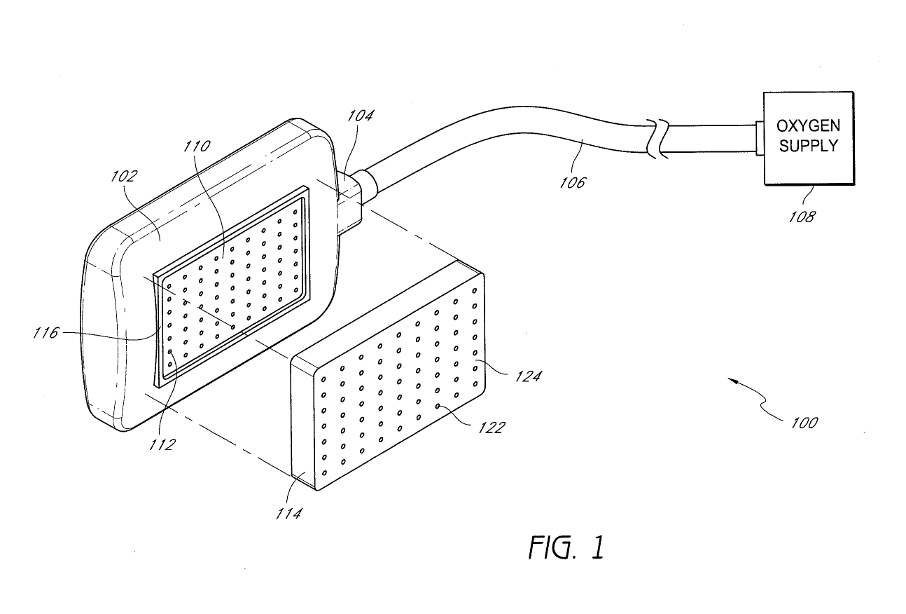

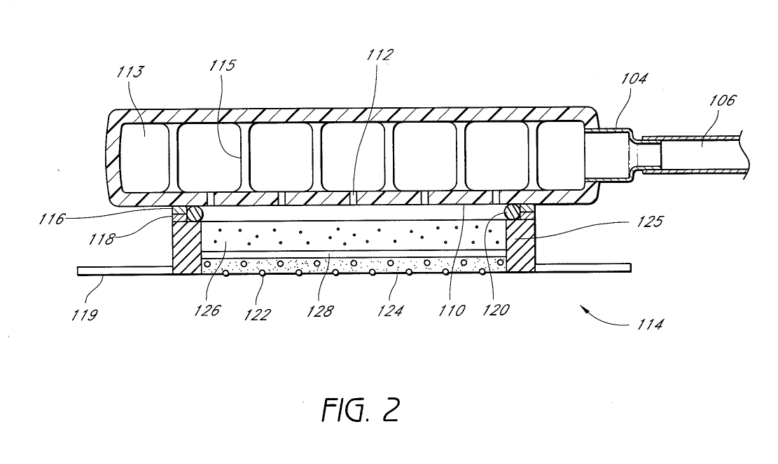

FIGS. 1 and 2 schematically show an embodiment of a wound therapy system 100 that includes a gas emitter, such as an oxygen dispenser 102. The gas emitter can include an inlet 104 configured to couple with a tube 106, which in turn can connect to a gas supply 108. The gas supply 108 can be an oxygen concentrator (e.g., a zeolite-based oxygen concentrator), a compressed oxygen tank, a hospital oxygen system, or any other source of therapeutic gas. In some embodiments, the oxygen supply can provide oxygen at a concentration of at least about 60% or at least about 90% and/or up to about 100%, or any other therapeutic concentration higher than the concentration of oxygen in atmospheric air (e.g., about 20%). In some embodiments, a flow controller (not shown) can be included on the oxygen dispenser 102, the tube 106, or the oxygen supply 108 for adjusting the rate of flow of oxygen. In some embodiments, the oxygen dispenser can be configured to release oxygen at an adjustable or fixed rate of at least about 0.5 liters per minute and/or less than or equal to about 100 liters per minute.

In some embodiments, the oxygen dispenser 102 can include an oxygen concentration adjuster (not shown) which allows the user to modify the amount of atmospheric air that is combined with the oxygen received through the inlet 104. The oxygen dispenser 102 can have a patient side 110 that is configured to face toward the wound and direct the oxygen in the direction of the wound. The patient side 110 may include a porous or fenestrated material or a membrane that allows the oxygen to pass through the patient side 110. In some embodiments, the patient side 110 or some other region of the gas emitter 102 is made of a material impermeable to oxygen and includes a number of holes 112 that allow the oxygen to escape from the oxygen dispenser 102. In some embodiments, the holes 112 can vary in size to achieve a substantially even distribution of oxygen across the patient side 100. For example, the holes nearer the inlet can be smaller in size than the holes further from the inlet. In some embodiment, the holes 112 can be equal sized. Internal flow regulating structure can be utilized to generally provide equal flow rates through the holes, such as tubing between each hole and the inlet 104 with generally equal lengths.

In some embodiments, the oxygen dispenser 102 can be formed from a flexible material (e.g., plastic or rubber) that allows the oxygen dispenser 102 to be molded, twisted, or bent to conform to the shape of the patient's body where the wound is located and/or to function as a comfortable cushion or pillow for the affected region. The wound therapy system can also include attachment members, such as straps, ties, elastic sleeves, elastic mitts, elastic enclosures, etc. to maintain the gas emitter 102 and/or pad 114 in close proximity to or in contact with the wound.

In some embodiments, the oxygen dispenser can be an air bladder. In some embodiments, the oxygen dispenser 102 can be made from a material that has enough rigidity that the sides of the oxygen dispenser are kept apart and the interior chamber 113 of the oxygen dispenser 102 does not collapse and block some of the holes 112 when pressure is applied (e.g., when positioned under a lying patient). In some embodiments, the oxygen dispenser 102 includes one or more support structures 115 located inside the internal chamber 113, to prevent the internal chamber 113 from collapsing when pressure is applied. In some embodiments, the oxygen dispenser 102 is made from a rigid material, such as a hard plastic or metal.

In some embodiments, the oxygen dispenser 102 can include one or more distribution structures (not separately shown) to direct the flow of oxygen inside the internal chamber 113. In some embodiments, the distribution structures are configured to provide a substantially uniform output of oxygen from the patient side 110 of the oxygen dispenser 102. The distribution structures may be integrated with the support structures 115 or they can be separate structures altogether. Various configurations of internal structures can be used to modify, adjust, or generally equalize air flow.

A therapeutic agent containing portion, such as a pad 114, can be disposed on the patient side 110. The containing portion can comprise a pouch, such as a mesh pouch closed on all sides, for containing one or more therapeutic agents in loose particulate form. In some embodiments, the containing portion can also be a topical portion configured to make contact with a wound. As used herein the term “pad” can also be applied generally to other types of containing portions.

The pad 114 can be removably attachable to the oxygen dispenser 102. As illustrated, the oxygen dispenser 102 can include a pad connector 116 which can be located along the perimeter of the patient side 110. The pad connector 116 can be configured to removably couple with a corresponding connector 118 on the pad 114. The connectors 116, 118 can use securing members, such as Velcro, snaps, clips, etc., to removably attach the pad 114 to the oxygen dispenser 102. In some embodiment, the pad 114 can include a weak adhesive that allows the pad 114 to be selectively attached to and detached from the oxygen dispenser, and the connector 116 on the oxygen dispenser can be omitted. In some embodiments, the pad 114 includes a seal 120 formed around the perimeter of the pad 114 which creates a seal against the patient side 110 of the oxygen dispenser 102 to prevent oxygen from escaping through the interface between the pad 114 and oxygen dispenser 102. In some embodiments, the seal 120 can be integrated as part of the connector 118. The pad 104 may also include a patient connector which can comprise one or more flaps 119 coated with adhesive configured to adhere to the skin surrounding the wound. Various other types of patient connectors (e.g., straps) can be used. In some embodiments, the patient connector (e.g., adhesive flaps 119) can be attached to the oxygen dispenser 102 rather than to the pad 114.

In some embodiments, the pad 114 can be a substrate and can include a topical therapeutic agent 122 for accelerating the healing of the wound. Although many of the embodiments discussed herein employ zeolite particles as a therapeutic agent 122 for accelerating wound healing, a variety of other therapeutic agents can be used instead of or in addition to zeolite particles. Exemplary materials that can be used include, but are not limited to, therapeutic agents, antibiotics, antifungal agents, antibacterial agents, antimicrobial agents, anti-inflammatory agents (including, for example, steroids such as cortisone, hydrocortisone, prednisone, prednisolone, methylprednisone, triamcinolone, fluoromethalone, dexamethasone, medrysone, betamethasone, loteprednol, fluocinolone, flumethasone, mometasone, testosterone, methyltestosterone, danazol, etc.; NSAIDs including ibuprofen, naproxen, salicylic acid, aspirin, etc.), analgesics such as acetominophen, antihistamines (e.g., cimetidine, chloropheniramine maleate, diphenahydramine hydrochloride, and promethazine hydrochloride), iodine, botanical agents, compounds containing silver ions or ions of other transition metals such as iron, gold, mercury, chromium, manganese, copper, nickel, palladium, platinum, and zinc, and many other materials known in the art or similar materials yet to be devised or used. In some embodiments, multiple therapeutic agents are applied to the pad 114.

In some embodiments, a hemostatic agent can be used as the therapeutic agent 122 to accelerate wound healing. For example, the hemostatic agent may be a molecular sieve material, a bioactive glass material, a mesoporous material, a clay mineral, or a combination of any of the foregoing. Other hemostatic materials can also be used in addition to or instead of the foregoing, such as ascorbic acid, tranexamic acid, rutin, thrombin, chitosan, fibrin, Factor VII or similar enzymes. Other hemostatic agents known in the art or yet to be devised can also be used. In some embodiments, the hemostatic agent has an extremely large ratio of volume to surface area. For example, a volume of about one teaspoon of some hemostatic agents can provide a surface area of about 50,000 square feet.

The molecular structure of some zeolites suitable for use in some embodiments is referred to as an “A-type” crystal, namely, one having a cubic crystalline structure that defines round or substantially round openings. Exemplary zeolites of this type include 5A, 4A, and 3A type zeolites. In some embodiments, the therapeutic agent can be a 5A type zeolite, although other zeolites can be used. Naturally occurring or synthetically produced zeolites can be used. Naturally occurring zeolites (e.g., analcite, chabazite, heulandite, natrolite, stilbite, thomosonite, and others) can be found as deposits in sedimentary environments as well as in other places. Synthetically produced zeolites can be produced, for example, by processes in which rare earth oxides are substituted by silicates, alumina, or alumina in combination with alkali or alkaline earth metal oxides. In some embodiments, the preferred zeolite is the 5A type, with the chemical structure 0.80 CaO : 0.20 Na₂O : 1 Al₂O₃: 2.0±0.1 SiO₂: ×H₂O.

In some embodiments, the zeolite (or other therapeutic agent) is used in a powder form, but granular, beaded, pellet, or other forms of zeolites can also be employed. Powdered zeolite can be obtained, for example, by grinding, crushing, rolling, or pulverizing coarser zeolite material. In some embodiments, larger particles of zeolite can be used to reduce the amount of surface area that is available for contact with blood or with the wound. Thus, the potency (e.g., rate of clotting) can be adjusted by varying the size of zeolite particles used. As is discussed in more detail below, it is sometime desirable to apply the zeolite particles to a non-bleeding wound or a wound with little bleeding, and in some cases limited absorption is desirable. However, in some embodiments, a high degree of absorption is desirable, such as when treating a wound with significant bleeding. In some embodiments, the oxygen dispenser 102 and pad 114 can be placed under a lying patient to treat a wound (e.g., a bedsore), and small particle sizes can be used so as to avoid patient discomfort. Therefore, a wide range or zeolite particle sizes can be used. For example, in some embodiments, the zeolite particles used can have a size of at least about 0.4 mm and/or less than about 1 mm.

In some embodiments, the therapeutic agent 122 can be incorporated into a substrate 124. The substrate 124 can be a porous web material configured to retain the therapeutic agent 122. Polymer materials can be used to form the web structures, and solid matrices can be useful where the therapeutic agent 122 particles reside bound to the surface of the polymer sheet. In some embodiments, the substrate 124 can be an open-cell foam having porosity throughout the substrate 124 to form a sponge structure. The substrate 124 may be in the form of a woven or non-woven natural, or woven or non-woven synthetic cloth. The substrate may take other forms such as gauze, paper (e.g., adsorbent media paper, polyethylene sheet paper, cellulosic paper, surgical grade kraft paper or Tyvek® artificial paper), films, permeable membranes (such as microporous membranes), and/or non-permeable membranes.

Generally, the different forms of substrates may comprise similar materials. Examples of substrate materials include natural materials comprising materials such as cellulose (e.g. cotton), silk, wool, hemp, etc.; natural fiber derivatives (such as oxidized cellulose, esterified cellulose, etherified cellulose, etc.); polymeric materials such as polyalkylene, polyethylene, polypropylene, etc; polyvinylesters such as polyvinylacetate; polyvinyl alcohol; polyesters; polyamides, including polyurethanes; polyacrylates such as polyacrylic acid, polyalkylacrylic acid, polyalkylacrylate (e.g. polymethacrylate) polyalkylacrylates (e.g. polymethylmethacrylate); polyalkylene oxides such as polyethylene oxide or polypropylene oxide; halogenated polymers such as polyvinylhalides (e.g. polyviny chloride), fluorinated polymers (e.g. polyvinylfluoride, polyvinylidenefluoride, polychlorotrifluoroethylene, polyfluoroethylenepropylene, etc.), perfluorinated polymers (e.g. polytetrafluoroethylene, perfluoroalkoxyethylene, etc.); and copolymers or blends thereof.

In some embodiments, the substrate 124 includes synthetic cloth substrates like Tyvek® and Gortex®. In some embodiments, the substrate 124 includes synthetic polymeric plastics such as Mylar® (polyethylene terephalate polyesters), polyethylene film, polypropylene film, polyethylene-polyamide laminated film, polyethylene-polyester laminated film, polypropylene-polyester laminated film, polyethylene-cellophane laminated film, polyethylene-stretched polypropylene laminated film, and combinations of the foregoing. In some embodiments, the substrate 124 can include a flexible, air permeable, high temperature resistant, and/or bacteria-impermeable material, which can be made of non-woven polyester layers (polymeric fibrous materials such as polypropylene or Reemay® polyester or Veratec® polyester). In some embodiments, the substrate 124 can also include a microporous membrane. The microporous membrane may be a hydrophobic fluoropolymer membrane such as microporous polytetrafluoroethylene, polyvinylfluoride, polyvinylidenefluoride, polychlorotrifluoroethylene, polyfluoroethylenepropylene, perfluoroalkoxyethylene and tetrafluoroethylene (TFE) copolymers, chlorotrifluoroethylene and ethylene copolymers, TFE and ethylene copolymers, and combinations of the foregoing. Other substrate materials known in the art or yet to be devised can also be used.

In some embodiments, the substrate 124 is made from a material that is permeable to air (e.g., an open cell foam), and the oxygen can pass through the material toward the wound. In some embodiments, the substrate 124 is not permeable to oxygen, but the substrate includes a number of holes (not shown) that allow the oxygen to pass through the substrate 124 and reach the wound. In some embodiments, a peripheral sealing layer 125 can extend along the side edges of the pad 114 to prevent the oxygen from escaping out the sides of the pad 114. In some embodiments, the peripheral sealing layer only partially covers the side edges of the pad 114 so that some air can escape, preventing pressure from building up near the wound. The pad may include other means for preventing pressure from building up near the wound, such as one-way valves (not shown), or release areas (not shown) made from an air permeable but bacteria impermeable material. In some embodiments, the peripheral sealing layer 125 comprises air permeable but bacteria permeable release areas.

Various methods can be used to apply the therapeutic agent 122 to the substrate 124. For example, zeolite particles (or other therapeutic agents) can be incorporated into the web structure of the substrate 124 during formation of the web, or they can be impregnated into the finished web by rolling or other impregnation methods known in the art or yet to be devised. In some embodiments, the therapeutic agent(s) can be adhesively or otherwise bonded to the substrate, such as with a binder, e.g., a water soluble polyol such as glycerol; a sugar alcohol such as sorbitol, erythritol, mannitol, lactitol, maltitol, etc.; a compound represented by a formula CH₂(OH)(CHOH)ₙCH₂OH, wherein n is 2, 3, 4, 5, 6, 7, or 8; a monosaccharide sugar such as glucose, mannose, galactose, etc.; a polymeric polyol such as polyvinyl alcohol; etc. The mechanism for adhesion between the zeolite particles and the substrate can be coulombic forces, a separate binding material (e.g., glycerin), or an additional therapeutic agent. For example, a biocompatible composition having properties that allow the composition to be retained on the substrate and to retain the therapeutic agent can be used as a binding agent. In some embodiments, the combination of binding agent and zeolite (or other therapeutic agent) can be smeared or otherwise applied to the surface of the substrate.

In embodiments using a cellulose or cellulose-based substrate, a zeolite-cellulose composite can be produced by impregnating the substrate with an aqueous solution of the zeolite. The substrate may be immersed in the aqueous solution, or an aqueous solution may be sprayed on the substrate or applied using a coating device. Other techniques can be used. One exemplary alternate technique for producing a zeolite-cellulose composite includes impregnating a cellulose substrate with an aqueous solution of an aluminum compound followed by immersing the aluminum-impregnated cellulose substrate in an aqueous solution of a silicon compound and a basic substance. In the alternative, a cellulose substrate can first be impregnated with an aqueous solution of a basic substance and then immersed in one of a silicon compound and an aluminum compound, followed by immersion of the base-impregnated substrate in the other of the silicon compound and the aluminum compound. Basic substances that may be used include, but are not limited to, mixed sodium, hydroxide, potassium hydroxide, and the like.

The therapeutic agent can be incorporated into a substrate made from a non-woven fibrous web of polymer material by a melt blowing technique. The polymer is melted and combined with the therapeutic agent and hot air. The melt is drawn into fine fibers which are cooled and collected as a web. Additional structure and/or processes for using topical agents and substrates are disclosed by U.S. Patent Publication No. 2008/0317831, the entirety of which is incorporated herein by reference. All structures and processes disclosed therein can be used in suitable embodiments of the inventions disclosed herein.

In some embodiments, other therapeutic agents can be incorporated into the substrate 124 by methods similar to those discussed above with regard to zeolites. In some embodiments, multiple therapeutic agents can be incorporated together at the same time, and in some embodiments different therapeutic agents can be applied at different times. For example one or more additional therapeutic agents can be mixed with, associated with, or incorporated into the zeolites before the zeolites are incorporated into the substrate 124. Many combinations of therapeutic agents are possible. In some embodiments, substrates 124 with different types of topical agents or concentrations thereof can be provided in sterilized packaging for attachment or removal from the gas emitter at different times, depending on the needs of different types of patients or a specific patient's changing needs.

In some embodiments, the therapeutic agent 122 is deposited throughout substantially the entire volume of the substrate 124 and, in some embodiments, throughout substantially the entire volume of the pad 114. In some embodiments, at least some particles of a topical agent (e.g., zeolite particles) protrude past the surface of the substrate 124 so that the particles of the topical agent directly contact the wound. As will be discussed in more detail below, in some cases, the application of zeolites to a wound can stimulate accelerated healing beyond the ability to quickly stop bleeding. Accordingly, in embodiments where the wound therapy system 100 is designed to treat a non-bleeding wound or a wound where a little bleeding is expected, the zeolites (or other topical agent) can be deposited on only the surface of the substrate.

The moisture content of the therapeutic agent particles may affect their effectiveness (e.g., absorption rate). In some embodiments, the desired moisture content can be reached by a drying and then re-hydrating of the particles. Alternatively, the particles can be fully saturated and subsequently dried to the desired moisture content level. The absorption of water by the zeolite causes an exothermic (heat-producing) reaction. In some embodiments, the heat can be a useful feature (e.g., to provide an elevated healing temperature or to ease discomfort in a wound). However, high levels of heat can be undesirable in some applications, such as treating a non-bleeding wound or applying zeolite to a wound for an extended period of time. As the moisture content of the zeolite increases, the less absorbent and the less exothermic the zeolite becomes. In some embodiments, the moisture content of the therapeutic agent may be at least about 5% by weight and/or less than or equal to about 15% by weight.

The particle size, concentration, and hydration of the zeolite can be adjusted to achieve the desired potency and comfort level for the patient. For example, a smaller particle size can be used to provide a pad which a patient can comfortably lie upon. To reduce the level of heat produced by the highly absorbent small zeolite particles, the hydration of the zeolite can be increased or the concentration can be reduced. In some embodiments, zeolite particles having an average size of at least about 0.4 mm and/or less than about 2.4 mm, and a moisture content of at least about 5% and/or less than or equal to about 15%, is applied to the substrate. In some embodiments, the pad 114 can be a container such as a mesh pouch that includes therein a hemostatic agent, such as QuikClot® sold by Z-Medica Corporation of Wallingford, Conn. Various embodiments of devices for the delivery of hemostatic agents are described in U.S. patent application Ser. Nos. 2006/0178609 and 2007/0104768, each of which is incorporated herein by reference in its entirety.

In some embodiments, the pad 114 is a single layer pad made up of the substrate 124. In some embodiments, pad 114 can have multiple layers, one of which is the substrate 124. The pad 114 can include a cushion layer 126 located between the substrate and the oxygen dispenser 102. The cushion layer 126 can be made from a compressive and resilient material (e.g., foam or gauze), enabling the pad 114 to conform to the shape of the patient's body where the wound is located so that the surface of the pad 114 and the therapeutic agent are kept in contact with the surface of the wound. The cushion layer 126 can also make it more comfortable for the patient to lie on the device during treatment, such as when treating a bedsore. The cushion layer 126 can be made of a porous material or it can include holes (not shown) to allow oxygen to pass through it. In some embodiments, the cushion layer 126 is made of the same material as the substrate 124 except that the cushion layer 126 does not incorporate a therapeutic agent or other wound treating agent. In some embodiments, the pad 114 includes a barrier layer 128 located between the substrate 124 and the cushion layer 126. In some embodiments, the barrier layer 128 can be made of an oxygen permeable but bacteria impermeable membrane. In some embodiments, the barrier layer 128 can be used during the incorporation of the therapeutic agent 122 into the substrate 124 to prevent the therapeutic agent 122 from extending into the cushion layer 126.

In some embodiments, especially those used for treating pressure ulcers, it can be desirable to avoid heavy weight-bearing contact between the wound and other surfaces. For this reason, one or more weight-bearing or contact surfaces can be provided on the periphery of the pad 114 that are positioned closer to the patient than the surface underlying the pad 114. In this way, when the gas emitter housing is brought near the patient's wound, the weight of or contact with the affected body part can be borne primarily by the interface between the weight-bearing surface(s) of the housing and a non-wounded surface of the patient, while still permitting the pad 114 to be positioned in close proximity or light contact with the wound itself In some embodiments, the weight bearing or contact surface of the gas emitter housing can be adjusted in how far it extends above the outer surface of the housing, and/or in how far it extends away from the periphery of the pad, to be useful in a wider array of patient applications (e.g., to accommodate different body parts or wound sizes).

The oxygen dispenser 102 and pad 114 can assume a variety of sizes and shapes. For example, different sizes and shapes can be used depending on the size and location of the wound to be treated. In some embodiments, the pad 114 can be a twelve inch by twelve inch square pad. In some embodiments, the pad 114 can be rectangular, or circular, or elliptical, or any number of other shapes. In some embodiments, the pad 114 has a thickness of at least about ⅛ of an inch and/or less than about 4 inches. In some embodiments, the pad 114 has a thickness of about one inch. The thickness of the pad 114 can be adjusted within or outside of these ranges for many reasons, such as to improve the desired properties for containing topical agents, to achieve a desired degree of cushioning, and/or to help in properly dispersing the gas.

The surface of the pad 114 may be planar as shown or it may be shaped to fit a particular area of the patient's body. For example, the pad 114 may be shaped to fit an elbow or a foot, etc. In some embodiments, the oxygen dispenser 102 and pad 114 can be configured to be portable and/or worn by a patient, for example using an attachment structure comprising an elastic sock, sleeve, or sheath. In some portable embodiments, the gas emitter may include a portable power supply (e.g., a rechargeable battery), and/or a portable gas supply (e.g., a compressed gas tank) or a gas generator.

The shapes and sizes of the various components can vary. For example, the oxygen dispenser 102 and pad 114 may be much thinner than as shown. In some embodiments, the oxygen dispenser 102 and pad 114 can have a combined thickness of at least about ⅛ of an inch and/or less than about four inches. In some embodiments, the oxygen dispenser 102 and the pad 114 have a combined thickness of about one inch. While FIGS. 1 and 2 show the pad 114 covering only a portion of the patient side 110 of the oxygen dispenser 102, it should be noted that other configurations are possible. For example, the pad 114 can cover substantially the entire patient side 110 of the oxygen dispenser so that the edges of the pad and the edges of the oxygen dispenser are about flush. Or the pad 114 can cover substantially all or all of the outer surface area of the gas emitter. In some embodiments, this configuration can provide a removable outer portion that can be discarded between uses, especially uses by different patients, while maintaining the underlying cleanliness and/or sterility of the gas emitter. The pad 114 or a portion thereof (such as the underlying surface in contact with the gas emitter) may comprise a hydrophobic material that permits passage of gas out of the gas emitter and through the pad 114, but that generally inhibits the passage of liquids onto or into the gas emitter. In some embodiments, the oxygen dispenser 102 covers only a portion of the pad 114.

The gas dispensing unit can also comprise a temperature regulator or adjuster. In some embodiments, a heater unit can increase the temperature and/or a cooling unit can diminish the temperature for improved healing and/or comfort. The temperature regulator or adjuster can include controls for modifying the temperature and/or sensors for measuring the patient's skin temperature and/or the ambient temperature. The system can also comprise a timer feature for determining the amount of time that the system has been applied to a patient. The time can include an audible or visual indicator or an automatic shut off after a specified period of time.

The oxygen dispenser 102 and pad 114 can be used in a variety of treatment methods. In some embodiments, the pad 114 is removably attachable to the oxygen dispenser 102, and the oxygen dispenser 102 can be reusable. After each use, the used pad can be removed from the oxygen dispenser 102 and discarded. In some embodiments, the oxygen dispenser 102 can be compatible with different types of interchangeable pads having different sizes or different shapes or different therapeutic agents applied to them. In some embodiments, the pad 114 can be worn by the patient as a patch such as by applying adhesive flaps 119 to the skin surrounding the wound. Then the oxygen dispenser can be connected and disconnected for periodic treatments of topical oxygen. In some embodiments, the oxygen dispenser 102 and pad 114 are not connected to the patient by adhesive flaps or straps or other means, and the oxygen dispenser 102 and pad 114 are merely placed on the wound. For example, the oxygen dispenser 102 and pad 114 can be placed under a lying patient to treat a bedsore. In some applications, it is desirable to apply the therapeutic agent 122 (e.g., zeolite) or the oxygen treatment to the wound for a relatively short amount of time (as discussed in more detail below). In these embodiments, the oxygen dispenser 102 and pad 114 can be held against a wound by a medical practitioner.

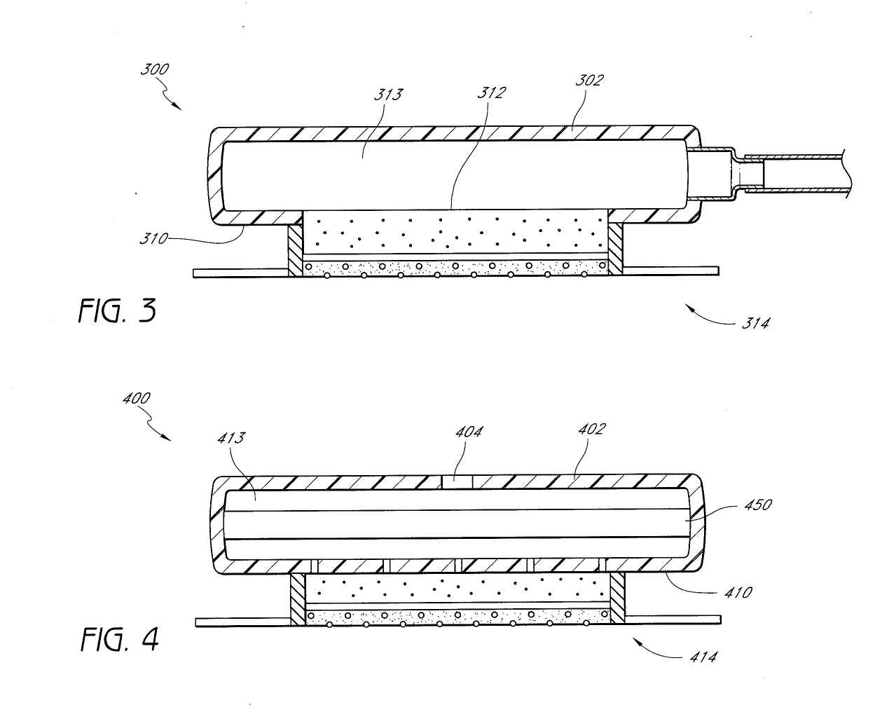

Turning now to FIG. 3, a wound treatment device 300 is shown that includes an oxygen dispenser 302 and a pad 314 that is integrated into the oxygen dispenser 302. The oxygen dispenser 302 and the pad 314 can have some features that are similar to or the same as those discussed above with regard to the oxygen dispenser 102 and pad 114. However, some features can be different. For example, connectors 116 and 118 can be omitted. The patient side 310 of the oxygen dispenser 302 has a large opening 312 and part of the pad 314 is disposed in the opening 312 so that the pad 314 is in direct contact with the interior chamber 313. In some embodiments the pad 313 can be secured to the oxygen dispenser 302 with an adhesive. In embodiments where the pad 314 is not removable from the oxygen dispenser 302, the wound treatment device 300 can be a single-use disposable unit.

Turning now to FIG. 4, a wound treatment device 400 is shown. The wound treatment device 400 can include a gas dispenser 402 that has a gas generator 450 contained within the interior chamber 413 for extracting oxygen from atmospheric air or some other source. In some embodiments, the gas dispenser is an oxygen dispenser. As illustrated, the oxygen dispenser 402 need not include an inlet configured to connect to an oxygen supply. Rather, the oxygen dispenser 402 includes at least one inlet 404 for taking in atmospheric air. A pad 414 can be attached to the patient side 410 of the oxygen dispenser 402. In some embodiments, the pad 414 can be secured directly to the oxygen dispenser 402 with an adhesive. The patient side 410 can extend between the pad 414 and the interior chamber 413 and holes 412 can allow oxygen to pass through the patient side 410 to the pad 414.

Because the wound treatment device 400 retrieves oxygen from the atmospheric air and need not be connected to an external oxygen supply, it is particularly suitable for use as a single-use bandage for mobile patients. For example, the treatment device 400 can be worn as a patch over a wound and can deliver both topical oxygen therapy and therapeutic agents (e.g., zeolites) to the wound to accelerate healing while the patient carries on normal activities.

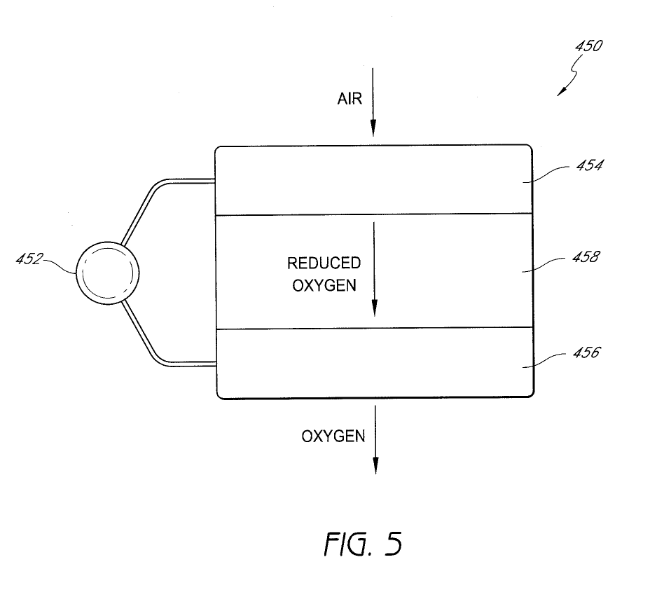

Turning now to FIG. 5, a gas generator, such as an oxygen generator 450 can produce a gas, such as oxygen, through an electrochemical process. In some embodiments, the oxygen generator includes a power supply 452 (e.g., a battery) electrically connected to two electrodes 454, 456. The electrodes 454, 456 can be separated by a permeable membrane 458. When the power supply 452 applies an electric current, the electrode 454 can act as a cathode while electrode 456 can act as an anode. The electrode 454 can be exposed to atmospheric air, and at electrode 454 a cathodic reaction takes place that combines the oxygen in the atmospheric air to form a chemical species (e.g., water, hydroxyl ions, peroxide, or superoxide). The voltage gradient created between the electrodes 454, 456 causes the chemical species containing the reduced oxygen to travel through the permeable membrane 458 to the electrode 456, where the chemical species are reconverted into oxygen. The oxygen is then directed toward the pad 114 and the underlying wound. Although the oxygen generator 450 described in FIG. 5 is an electrochemical oxygen generator, other types of oxygen generators that are known in the art or yet to be devised can be used.

The electrodes 454, 456 can be a permeable electrically conductive mesh or coating applied to the membrane 458. A variety of materials can be used that will convert oxygen from the atmospheric air to reduced oxygen in the chemical species. The permeable membrane 458 can be permeable to the specific chemical species produced by the electrode 454. A variety of materials can be used to form the electrodes 454, 456 and the permeable membrane 458 and a variety of chemical species can be used to transport the reduce oxygen across the permeable membrane 458.

The power supply 452 can be, for example, a battery, such as a zinc/air battery. In some embodiments, the power supply 452 can be configured to have a predetermined lifespan corresponding to the length of desired topical oxygen treatment. In some embodiments, the oxygen dispenser 402 can be a disposable unit, wherein the short term battery is activated by exposing the oxygen dispenser 402 to air. In some embodiments, the power supply can be toggled on and off to start and stop the generation of oxygen. In some embodiments, the power supply can be a replaceable unit, or an external power supply.

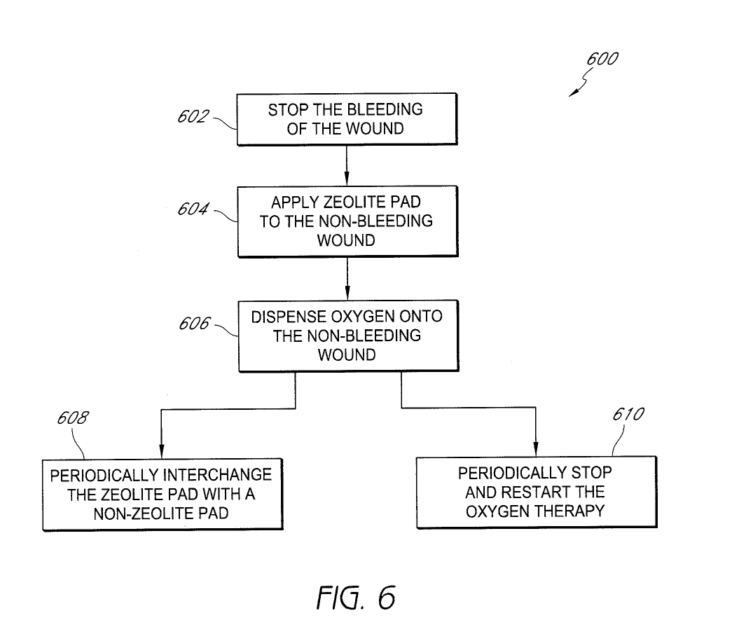

FIG. 6 describes an exemplary method 600 of treating a wound to accelerate the healing of the wound. At block 602 the bleeding of the wound is stopped. In some embodiments, zeolites or other therapeutic agents can be used to stop the bleeding. In some embodiments, the wound is a non-bleeding wound or the bleeding of the wound has stopped prior to treatment, and block 602 can be skipped. In some embodiments, a wound treatment device having an oxygen dispenser and a pad with therapeutic agents (e.g., zeolites) can be used to stop the bleeding at block 602. At block 604 a pad having zeolites incorporated therein is applied to the wound. Although this embodiment is described with regard to using zeolites, other therapeutic agents can be used (e.g., therapeutic agents). In some embodiments, the zeolite pad can be attached to an oxygen dispenser as described above. In embodiments where a zeolite pad was used to stop the bleeding at block 602, the same zeolite pad can be left on the wound or it can be replaced with a fresh pad. In some embodiments, the zeolite pad can be placed under a patient (e.g., between the patient and a bed) to treat a wound (e.g., a bedsore), and the weight of the patient pressing down on the pad can press the zeolite particles onto the wound and surrounding skin. At block 606, oxygen is dispersed onto the wound. In some embodiments, the oxygen is dispersed through the zeolite pad. In some embodiments, the zeolite pad is removed or replaced with a non-zeolite pad before oxygen is dispersed onto the wound.

In the illustrated example, blocks 608 and 610 show steps in which the zeolite and topical oxygen treatments continue periodically. At block 608 the zeolite pad is periodically interchanged with a pad that does not have zeolite particles incorporated therein. In some embodiments, the non-zeolite pad can include other therapeutic agents as described above. In some embodiments, a new pad is used each time a replacement is made. At block 610, the topical oxygen therapy is periodically started and stopped. In some embodiments the topical oxygen therapy can be stopped and started by respectively attaching and detaching an oxygen dispenser from the pad covering the wound. In some embodiments the oxygen dispenser can remain connected to the pad and the treatment is stopped and started by merely closing and opening a connection to the oxygen supply. In some embodiments, one or both of blocks 608 and 610 can be omitted. For example, in some embodiments, a wound can be treated with a one-time application of the zeolite pad and oxygen therapy.

In some embodiments, the zeolite pad can be applied to the wound on a substantially constant basis (except when changing the pad) while the oxygen therapy is be applied periodically. In some embodiments, the oxygen therapy can be applied on a substantially constant basis while the zeolite pad is applied to the wound periodically. In some embodiments, the treatments can alternate so that both zeolites and oxygen are applied to the wound periodically, but not at the same time. In some embodiments, the oxygen therapy can be omitted entirely, so that healing of the wound is accelerated by the application of zeolites without oxygen being applied to the wound. In some embodiments, the oxygen therapy and zeolite therapy can be applied at different rates so that at times the treatments overlap but at other times they do not.

In some embodiments the zeolite pad and/or topical oxygen therapy can be applied to the wound for less than about one hour or less than about four hours. In some embodiments, the zeolite pad and/or topical oxygen therapy can be applied once each day, or more than once each day (e.g., once every hour, every four hours, every six hours, etc.), or less than once per day (e.g., once every two days, every three days, etc.). In some embodiments, the timing and frequency of the treatments can vary depending on the severity of the wound, the location of the wound, and the degree of healing. For example, as the wound heals, the treatments can be applied for less time and/or less frequently. In some embodiments, the zeolite pad and/or topical oxygen therapy can be applied to the wound for short periods of time (e.g., less than about five minutes, etc.) but with high frequency (e.g., at least about once every hour). In some embodiments, the zeolite pad and/or topical oxygen therapy can be applied to the wound on a substantially constant basis. In some embodiments, the scabbed surface of the wound can be loosened, scraped, moistened, cleaned, debrided (surgically, mechanically, and/or chemically) or removed (partially, substantially completely, or completely) before applying the pad and/or gas treatment to the wound or in between applications of the pad and/or gas treatment to the wound.

Some of the embodiments discussed herein describe the use of zeolites to accelerate the healing of a wound. The wound healing process can be categorized into three phases: (1) the inflammatory phase; (2) the proliferative phase; and (3) the remodeling phase. These phases are generally sequential but they can overlap in time to some degree. The inflammatory phase can range from the immediate infliction of the wound to two to five days and includes events such as hemostasis; phagocytosis of bacteria, debris, and damaged tissue; and release of blood clotting factors (e.g., Factor VIII, Factor IX, and Factor XI) that cause platelets to aggregate, thereby inducing the proliferative phase. The proliferative phase can range from about two days to about three weeks and includes events such as growth of new blood vessels, collagen deposition, new tissue formation, and wound contraction. The remodeling phase can range from about three weeks to about two years during which time the tissue is reinforced and strengthened. For example, during the remodeling phase type III collagen is replaced with the stronger type I collagen.

Topical agents, such as zeolite-based therapeutic agents, can promote accelerated wound healing when applied to a wound. Topical agents can facilitate hemostasis, which in turn accelerates the clotting cascade and platelet aggregation. By accelerating the events of the inflammatory phase, the agents allow the proliferative phase to begin sooner. Also, by reducing blood loss the agents reduce the risk of infection and other complications that can delay wound healing. Zeolite-based therapeutic agents can also accelerate the proliferative phase of the healing process. The agents cause local inflammation on and around the wound site which increases fibroblast deposition and wound contraction. Thus, tissue can be re-epithelized at a faster rate than if no therapeutic agent was applied. The application of zeolite particles to a wound during the healing process can also result in reduced scaring and improved the quality of new tissue grown during the healing.

A wide variety of other variations are possible. Components can be added, removed, and/or rearranged. It should be noted that many features of the embodiments disclosed are interchangeable. For example, a wound treatment device that employs an oxygen generator (as described above with regard to FIG. 4) can have a pad secured to the patient side of the oxygen dispenser (as shown in FIG. 4), integrated into the oxygen dispenser 402 (as shown in FIG. 3), or removably attachable to the oxygen dispenser (as shown in FIGS. 1 and 2). Many other features described in connection with the various embodiments disclosed herein are also interchangeable. Similarly, in any method or process disclosed herein, steps or operations can be add, removed, and/or rearranged.

Reference throughout this specification to “some embodiments,” “certain embodiments,” or “an embodiment” means that a particular feature, structure or characteristic described in connection with the embodiment is included in at least some embodiments. Thus, appearances of the phrases “in some embodiments” or “in an embodiment” in various places throughout this specification are not necessarily all referring to the same embodiment and may refer to one or more of the same or different embodiments. Furthermore, the particular features, structures or characteristics may be combined in any suitable manner, as would be apparent to one of ordinary skill in the art from this disclosure, in one or more embodiments.

As used in this application, the terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Similarly, it should be appreciated that in the above description of embodiments, various features are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Rather, inventive aspects lie in a combination of fewer than all features of any single foregoing disclosed embodiment.

Although the inventions presented herein have been disclosed in the context of certain preferred embodiments and examples, it will be understood by those skilled in the art that the inventions extend beyond the specifically disclosed embodiments to other alternative embodiments and/or uses of the inventions and obvious modifications and equivalents thereof. Thus, it is intended that the scope of the inventions herein disclosed should not be limited by the particular embodiments described above.

1. A device for treating a wound, the device comprising:
a gas permeable pad configured to be placed in proximity to a wound, the gas permeable pad comprising a therapeutic agent; and
a gas dispenser disposed adjacent to the gas permeable pad, the gas dispenser configured to direct a gas through the gas permeable pad toward the wound.

2. The device of claim 1, wherein the gas dispenser is configured to direct a therapeutic concentration of oxygen through the gas permeable pad.

3. The device of claim 1, wherein the gas permeable pad comprises a material that is permeable to the gas.

4. The device of claim 1, wherein the gas permeable pad comprises a plurality of holes extending therethrough to allow the gas to pass through the gas permeable pad.

5. The device of claim 1, wherein the therapeutic agent is a molecular sieve material.

6. The device of claim 5, wherein the therapeutic agent comprises zeolite particles.

7. The device of claim 6, wherein the zeolite particles are granular zeolite particles.

8. The device of claim 6, wherein the zeolite particles have an average size greater than or equal to about 0.4 mm and less than or equal to about 2.4 mm.

9. The device of claim 6, wherein the zeolite particles have an average moisture content between about 5% and 15% by weight.

10. The device of claim 1, wherein the therapeutic agent is applied to a surface of the gas permeable pad configured to face the wound.

11. The device of claim 1, wherein the gas dispenser comprises an inlet configured to connect to an external gas supply.

12. The device of claim 11, wherein the gas dispenser comprises a plurality of outlets and one or more distribution structures contained within the gas dispenser, the distribution structures configured to affect the distribution of the gas through the outlets.

13. The device of claim 2, wherein the gas dispenser comprises an inlet for taking in atmospheric air and an oxygen generator contained within the gas dispenser, the oxygen generator configured to extract the oxygen from the atmospheric air and direct the oxygen toward the gas permeable pad.

14. The device of claim 1, wherein the gas permeable pad is removably attachable to the gas dispenser.

15. The device of claim 1, wherein the gas permeable pad is secured to the gas dispenser and the device is a single-use disposable unit.

16. The device of claim 1, wherein the gas dispenser comprises a flexible material and the gas dispenser also comprises one or more support structures disposed inside the gas dispenser, the support structures configured to prevent the gas dispenser from collapsing when pressure is applied to the gas dispenser.

17. A method of treating a wound on a patient, comprising:
applying a gas permeable pad to the wound, the gas permeable pad comprising a therapeutic agent, such that the therapeutic agent is configured to be placed in direct contact with the wound; and
dispersing a gas through the gas permeable pad and onto the wound.

18. The method of claim 17, wherein the gas comprises a therapeutic concentration of oxygen.

19. The method of claim 17, wherein the wound is a non-bleeding wound.

20. The method of claim 17, wherein the therapeutic agent comprises zeolite particles.

21. The method of claim 17, wherein applying a gas permeable pad to the wound comprises placing the gas permeable pad directly under the patient such that the weight of the patient presses the therapeutic agent onto the wound.
